(19) United States
(12) Patent Application Publication
(10) Pub. No.: US 2022/0356417 A1
(43) Pub. Date: Nov. 10, 2022

(54) DEOXYRIBONUCLEASE USES IN DETERGENT COMPOSITION

(71) Applicant: Novozymes A/S, Bagsvaerd (DK)

(72) Inventors: Yao Wang, Beijing (CN); Lise Munch Mikkelsen, Roedovre (DK); Henrik Lund, Copenhagen (DK)

(73) Assignee: Novozymes A/S, Bagsvaerd (DK)

(21) Appl. No.: 17/762,689

(22) PCT Filed: Sep. 29, 2020
(86) PCT No.: PCT/CN2020/118746

§ 371 Date: Mar. 22, 2022

(30) Foreign Application Priority Data
Sep. 29, 2019 (CN) PCT/CN2019/109012

(51) Int. Cl. C11D 3/386; C11D 11/00; C11D 3/00; C12N 9/22; C12N 9/42

(52) U.S. Cl. CPC C11D 3/38636; C11D 11/0017; C11D 3/0036; C12N 9/22; C12Y 302/01004; C12N 9/2437

(57) ABSTRACT

Detergent compositions comprising a deoxyribonuclease (DNase) and uses of DNase in the detergent are provided. The use of DNase in the detergent provides improved whiteness and anti-redeposition effects, and may reduce the use level of conventional anti-redeposition polymers.

### REFERENCE TO A SEQUENCE LISTING

This application contains a Sequence Listing in computer readable form, which is incorporated herein by reference.

### FIELD OF THE INVENTION

The present invention concerns a detergent composition comprising a deoxyribonuclease (DNase). It further concerns a laundering method and the use of DNase in detergent compositions.

### BACKGROUND OF INVENTION

Detergent compositions are well known to include a large number of ingredients, offering particular functionality throughout the cleaning process. However, some detergent ingredients have faced scrutiny due to potential environmental concerns. It is desirable to provide alternatives that are both safe from a manufacturability and consumer point of view, while maintaining compatibility with other detergent ingredients. In addition, the consumer benefits and performance effects must be maintained.

Greying, dinginess and yellowing of garments are concerns from the customer point of view, and currently polymer products such as carboxymethyl cellulose, polyacrylic acid, copolymers of maleic acid/acrylic acid and other modified polymers are developed to deal with the issue by preventing the deposition of particles on garments during wash.

EP 3 476 935 A1 (Procter & Gamble) discloses detergent compositions comprising DNase variants, said compositions being suitable for use in cleaning processes.

WO 2014/087011 (Novozymes) discloses a detergent composition comprising a DNase and the use of DNase for reducing malodour from laundry, for anti-redeposition and for maintaining or improving the whiteness of a textile.

WO 2015/155350 A1 (Novozymes) discloses a detergent and a pharmaceutical composition comprising a DNase, wherein the DNase is obtained from a fungal source.

WO 2017/001472 A1 (Novozymes) discloses a method for laundering a textile, the use of a DNase and a detergent composition comprising a DNase.

WO 2018/011277 A1 (Novozymes) discloses DNase variants suitable for use in cleaning processes and detergent compositions.

### SUMMARY OF THE INVENTION

The present invention relates to the use in detergent of a polypeptide having DNase activity for maintaining or improving whiteness of an item during a wash cycle in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, wherein the item is a textile. The invention further relates to use in detergent of a polypeptide having DNase activity for maintaining or improving whiteness of an item during a wash cycle as a reduction or even replacement for a a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof. The invention relates to use in detergent of a polypeptide having DNase activity for preventing, reducing or removing redeposition of soil to an item during a wash cycle in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, wherein the item is a textile.

The invention also relates to a detergent composition comprising a polypeptide having deoxyribonuclease (DNase) activity and a detergent adjunct ingredient, provided that the composition comprises less than 1%, e.g., less than 0.8%, less than 0.7%, less than 0.6%, less than 0.5%, less than 0.4%, less than 0.3%, less than 0.2%, less than 0.1%, less than 0.05%, less than 0,025% by weight of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.

The invention relates to a method for laundering an item, which method comprises the steps of: exposing an item to a wash liquor comprising a polypeptide having DNase activity or a detergent composition comprising the polypeptide in the absence of a copoly(acrylic acid/maleic acid) polymer, a polyacrylate polymer, a homopolymer of acrylic acid, carboxymethyl cellulose, methyl cellulose, and combinations thereof; completing at least one wash cycle; optionally adding additional soiling; and optionally rinsing the item, wherein the item is a textile. In an embodiment, the laundering method with the polypeptide having DNase activity provides the same or better whiteness of the item compared to a laundering method performed with a detergent composition including a polyacrylic acid, a modified polyacrylic acid polymer, a modified polyacrylic acid copolymer, a maleic acid-acrylic acid copolymer, carboxymethyl cellulose, cellulose gum, methyl cellulose, and combinations thereof.

### Definitions

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Bacterial: The term “bacterial” in relation to polypeptide (such as an enzyme, e.g. a DNase) refers to a polypeptide encoded by and thus directly derivable from the genome of a bacteria, where such bacteria has not been genetically modified to encode said polypeptide, e.g. by introducing the encoding sequence in the genome by recombinant DNA technology. In the context of the present invention, the term “bacterial DNase” or “polypeptide having DNase activity obtained from a bacterial source” or “polypeptide is of bacterial origin” thus refers to a DNase encoded by and thus directly derivable from the genome of a bacterial species, where the bacterial species has not been subjected to a genetic modification introducing recombinant DNA encoding said DNase. Thus, the nucleotide sequence encoding the bacterial polypeptide having DNase activity is a sequence naturally in the genetic background of a bacterial species. A sequence encoding a bacterial polypeptide having DNase activity may also be referred to a wildtype DNase (or parent DNase). Bacterial polypeptide having DNase activity includes recombinant produced wild types. In a further aspect, the invention provides polypeptides having DNase activity, wherein said polypeptides are substantially homologous to a bacterial DNase. In the context of the present invention, the term “substantially homologous” denotes a polypeptide having DNase activity which is at least 80%, preferably at least 85%, more preferably at least 90%, more preferably at least 95%, even more preferably at least 96%, 97%, 98%, and most preferably at least 99% identical to the amino acid sequence of a selected bacterial DNase.

Cellulolytic enzyme or cellulase: The term “cellulolytic enzyme” or “cellulase” means one or more (e.g., several) enzymes that hydrolyze a cellulosic material. Such enzymes include endoglucanase(s) (e.g. EC 3.2.1.4), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N21 filter paper, microcrystalline cellulose, bacterial cellulose, algal cellulose, cotton, pretreated lignocellulose, etc. The most common total cellulolytic activity assay is the filter paper assay using Whatman N21 filter paper as the substrate. The assay was established by the International Union of Pure and Applied Chemistry (IUPAC) (Ghose, 1987, Pure Appl. Chem. 59: 257-68).

Color difference (L value): A Lab color space is a color-opponent space with dimension L for lightness. L value, L* represents the darkest black at L*=0, and the brightest white at L*=100. In the context of the present invention L value is also referred to as color difference.

Detergent adjunct ingredient: The detergent adjunct ingredient is different to the DNAse of this invention. The precise nature of these additional adjunct components, and levels of incorporation thereof, will depend on the physical form of the composition and the nature of the operation for which it is to be used. Suitable adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, s, s, brighteners, suds suppressors, dyes, perfumes, structure elasticizing agents, fabric softeners, carriers, hydrotropes, builders and co-builders, fabric hueing agents, anti-foaming agents, dispersants, processing aids, solvents, and/or pigments.

Detergent Composition: The term “detergent composition” refers to compositions that find use in the removal of undesired compounds from items to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, bar, or spray compositions) and includes, but is not limited to, detergent compositions (e.g., liquid and/or solid laundry detergents and fine fabric detergents; fabric fresheners; fabric softeners; laundry boosters; and textile and laundry pre-spotters/pre-treatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases and mannanases, or any mixture thereof), and/or detergent adjunct ingredients such as surfactants, builders, chelators or chelating agents, bleach system or bleach components, polymers (as set forth herein), fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, bluing agents and fluorescent dyes, antioxidants, and solubilizers.

DNase (deoxyribonuclease): The term “DNase” means a polypeptide with DNase activity that catalyzes the hydrolytic cleavage of phosphodiester linkages in the DNA backbone, thus degrading DNA. For purposes of the present invention, DNase activity is determined according to the procedure described in the Assay I. In one aspect, the polypeptides of the present invention have at least 20%, e.g., at least 40%, at least 50%, at least 60%, at least 70%, at least 80%, at least 90%, at least 95%, or at least 100% of the DNase activity of the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 14. In one embodiment, the polypeptides of the present invention have improved DNAse activity, e.g. such that the DNAse activity of the polypeptide is at least 105%, e.g., at least 110%, at least 120%, at least 130%, at least 140%, at least 160%, at least 170%, at least 180%, or at least 200% with reference to the DNase activity of the mature polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 14.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Also included is the maintenance of whiteness, e.g., the prevention of greying or dullness. Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibres from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Fragment: The term “fragment” means a polypeptide having one or more (e.g., several) amino acids absent from the amino and/or carboxyl terminus of a mature polypeptide or domain; wherein the fragment has DNase activity.

Fungal: In the context of the present invention the term “fungal” in relation to polypeptide (such as an enzyme, e.g. a DNAse) refers to a polypeptide encoded by and thus directly derivable from the genome of a fungus, where such fungus has not been genetically modified to encode said polypeptide, e.g. by introducing the encoding sequence in the genome by recombinant DNA technology. In the context of the present invention, the term “fungal DNAse” or “polypeptide having DNAse activity obtained from a fungal source” thus refers to a DNAse encoded by and thus directly derivable from the genome of a fungal species, where the fungal species has not been subjected to a genetic modification introducing recombinant DNA encoding said DNAse. Thus, the nucleotide sequence encoding the fungal polypeptide having DNAse activity is a sequence naturally in the genetic background of a fungal species. The fungal polypeptide having DNAse activity encoding by such sequence may also be referred to a wildtype DNAse (or parent DNAse). In a further aspect, the invention provides polypeptides having DNase activity, wherein said polypeptides are substantially homologous to a fungal DNase. In the context of the present invention, the term “substantially homologous” denotes a polypeptide having DNase activity which is at least 80%, preferably at least 85%, more preferably at least 90%, more preferably at least 95%, even more preferably at least 96%, 97%, 98%, and most preferably at least 99% identical to the amino acid sequence of a selected fungal DNase. The polypeptides being substantially homologous to a fungal DNase may be included in the detergent of the present invention and/or be used in the methods of the present invention.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved wash performance: The term “improved wash performance” is defined herein as an enzyme displaying an increased wash performance in a detergent composition relative to the wash performance of same detergent composition without the enzyme e.g. by increased stain removal or less redeposition. The term “improved wash performance” includes wash performance in laundry.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Malodor: The term “malodor” means an odor which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly. One way of measuring the ability of an item to adhere malodor is by using Assay II disclosed herein.

Mature polypeptide: The term “mature polypeptide” means a polypeptide in its final form following translation and any post-translational modifications, such as N-terminal processing, C-terminal truncation, glycosylation, phosphorylation, etc.

Mature polypeptide coding sequence: The term “mature polypeptide coding sequence” means a polynucleotide that encodes a mature polypeptide having DNase activity.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Remission value: Wash performance is expressed as a Remission value of the stained swatches. After washing and rinsing the swatches are spread out flat and allowed to air dry at room temperature overnight. All washes swatches are evaluated the day after the wash. Light reflectance evaluations of the swatches are done using a Macbeth Color Eye 7000 reflectance spectrophotometer with very small aperture. The measurements are made without UV in the incident light and remission at 460 nm is extracted.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment).

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EM-BOSS: The European Molecular Biology Open Software Suite, Rice et al., 2000, supra), prefer-ably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment).

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and toweling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well. In the context of the present invention, the term “textile” also covers fabrics.

Variant: The term “variant” means a polypeptide having same activity as the parent enzyme comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In the context of the present invention, a variant of an identified DNAse has the enzymatic activity of the parent, i.e. the capacity of catalyzing the hydrolytic cleavage of phosphodiester linkages in the DNA backbone (deoxyribonuclease activity). In one embodiment, the deoxyribonuclease activity of the variant is increased with reference to the parent DNAse, e.g. the mature polypeptide of SEQ ID NO: 2.

Wash cycle: The term “wash cycle” is defined herein as a washing operation wherein textiles are immersed in the wash liquor, mechanical action of some kind is applied to the textile in order to release stains and to facilitate flow of wash liquor in and out of the textile and finally the superfluous wash liquor is removed. After one or more wash cycles, the textile is generally rinsed and dried.

Wash liquor: The term “wash liquor” is defined herein as the solution or mixture of water and detergent components optionally including the enzyme of the invention.

Wash time: The term “wash time” is defined herein as the time it takes for the entire washing process; i.e. the time for the wash cycle(s) and rinse cycle(s) together.

Whiteness: The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

### Sequence Overview

SEQ ID NO: 1 is a DNase obtained from Aspergillus oryzae.

SEQ ID NO: 2 is a DNase obtained from Bacillus licheniformis.

SEQ ID NO: 3 is a DNase obtained from Bacillus subtilis.

SEQ ID NO: 4 is a DNase obtained from Serratia marcescens.

SEQ ID NO: 5 is a DNase obtained from Bacillus idriensis.

SEQ ID NO: 6 is a DNase isolated from Bacillus cibi.

SEQ ID NO: 7 is a DNase obtained from Bacillus horikoshii.

SEQ ID NO: 8 is a DNase obtained from Bacillus sp.

SEQ ID NO: 9 is a DNase obtained from Bacillus sp.

SEQ ID NO: 10 is a cellulase obtained from Humicola insolens.

SEQ ID NO: 11 is a cellulase obtained from Bacillus akibai.

SEQ ID NO: 12 is a cellulase obtained from Paenibacillus polymyxa.

SEQ ID NO: 13 is a cellulase obtained from Melanocarpus albomyces.

SEQ ID NO: 14 is a DNase obtained from Aspergillus oryzae.

### DETAILED DESCRIPTION OF THE INVENTION

The inventors have found that polypeptides having deoxyribonuclease (DNase) activity can be used for preventing the deposition of particles on garments during wash, even in the absence of typical polymers found in liquid and powder detergent systems. Surprisingly, the present inventors have found that even in the absence of a conventional antiredeposition polymer, the DNase can act, e.g., by effectively removing body soil on worn clothes, as well as by causing less adherence of particles in wash, leading to excellent anti-greying performance. The present inventors have discovered that polypeptides having DNase activity can replace polymer regarding as having anti-redeposition benefit in detergent formulation.

As demonstrated in Example 1, while conventional antiredeposition polymers show benefit on textile in wash, polypeptides having DNase activity can show competitive or even better effect.

Accordingly, in an embodiment, the present invention concerns the use of a polypeptide having DNase activity for maintaining or improving whiteness of an item in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.

In an embodiment, the present invention concerns the use of a polypeptide having DNase activity for preventing, reducing, or removing redeposition of a soil to an item during a wash cycle conducted in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, wherein the item is a textile. When the soil does not adhere to the item, the item appears cleaner.

In one embodiment the present invention is directed to a detergent composition comprising a polypeptide having DNase activity and a detergent adjunct ingredient, wherein the composition comprises less than 1%, e.g., less than 0.8%, less than 0.7%, less than 0.6%, less than 0.5%, less than 0.4%, less than 0.3%, less than 0.2%, less than 0.1%, less than 0.05%, less than 0,025% by weight of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.

The invention further concerns a method for laundering an item, which method comprises the steps of:
a) exposing an item to a wash liquor comprising a polypeptide having DNase activity or a detergent composition comprising the polypeptide in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof;
b) completing at least one wash cycle;
c) optionally adding additional soiling; and
d) optionally rinsing the item,

wherein the item is a textile.

In an embodiment, the laundering method with the polypeptide having DNase activity provides the same or better whiteness of the item compared to a laundering method performed with a detergent composition including a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.

The pH at 25° C. of the liquid solution is in the range of 1 to 11, such as in the range 5.5 to 11, such as in the range of 7 to 9, in the range of 7 to 8 or in the range of 7 to 8.5. The pH of a powder detergent may be measured as 1 g/L in demineralized water and is preferably in the range of 1-12; such as 5.5-11.5; such as 7.5-11.5; such as 8-11.

The wash liquor may have a temperature in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C. or in the range of 20° C. to 30° C. In one embodiment the temperature of the wash liquor is 30° C.

In one embodiment of the invention, the method for laundering an item further comprises draining of the wash liquor or part of the wash liquor after completion of a wash cycle. The wash liquor can then be re-used in a subsequent wash cycle or in a subsequent rinse cycle. The item may be exposed to the wash liquor during a first and optionally a second or a third wash cycle. In one embodiment the item is rinsed after being exposed to the wash liquor. The item can be rinsed with water or with water comprising a conditioner.

A polypeptide having DNase activity or a deoxyribonuclease (DNase) is any enzyme that catalyzes the hydrolytic cleavage of phosphodiester linkages in the DNA backbone, thus degrading DNA. The two terms polypeptide having DNase activity and DNase are used interchangeably.

A DNase suitable for use in the methods and compositions of the invention is preferably a microbial DNase e.g. such as a Bacillus or fungal DNase. Preferably the bacillus DNase is selected from the group consisting of polypeptides comprising the amino acid sequences shown in SEQ ID NO: 2, 3, 5, 6, 7, 8, 9 and 14 or polypeptides having at least 60% identity, such as at least 65%, at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, or 100% sequence identity hereto.

Preferably the fungal DNase is a polypeptide comprising the amino acid sequences shown in SEQ ID NO: 1 or a polypeptide having at least 60% identity, such as at least 65%, at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, or 100% sequence identity hereto.

In another preferred embodiment the fungal DNase is a polypeptide comprising the amino acid sequences shown in SEQ ID NO: 14 or a polypeptide having at least 60% identity, such as at least 65%, at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, or 100% sequence identity hereto.

A suitable DNase may also be the Serratia marcescens DNase described in WO 201198579 and shown in SEQ ID NO: 4 or a polypeptide having at least 60% identity, such as at least 65%, at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99%, or 100% sequence identity hereto.

The DNase useful according to the present invention may be present in a detergent composition, the detergent composition may comprise at least 0.00002% DNase protein, preferably at least 0,000005%, 0,000001%, 0,00005%, 0,00001%, 0,0005%, 0,0001%, 0,005%, 0.001%, 0,002%, 0,003%, 0,004%, 0,005%, 0,006%, 0,008%, 0.01%, 0.02%, 0.03%, 0.05%, 0.1%, 0.2%, 0.3%, 0.4%, 0.6%, 0.7%, 0.8%, 0.9% or 1.0% of DNase protein as weight percent of the detergent composition.

The DNase useful according to the present invention can be added as formulated enzyme in an amount between 0,000002% to 10% as weight percent of the detergent composition. The DNase can be added as formulated enzyme in an amount between 0,00002% to 10%, such as 0,0002% to 5%, such as 0,002% to 5%, such as 0,002% to 3%, such as 0.02% to 3%, or even 0.05%, 0.1%, 0.15%, 0.2%, 0.25%, 0.3%, 0.35%, 0.4%, 0.45%, 0.5%, 0.6%, 0.7%, 0.8%, 0.9%, 1%, 1.5%, 2%, 2.5%, or 3% as weight percent of the detergent composition.

A DNase suitable in the present invention may be obtained from Aspergillus, for example from Aspergillus oryzae. A DNase suitable in the present invention may also be obtained from Bacillus, for example from Bacillus licheniformis, Bacillus subtilis, Bacillus horikoshii, Bacillus idriensis, Bacillus cibi and Bacillus sp.

In an embodiment, the present invention relates to a DNase obtained from Aspergillus in particular Aspergillus oryzae. In an embodiment, the present invention relates to a DNase polypeptide comprising the amino acid sequence of SEQ ID NO: 1 or SEQ ID NO: 14 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 1 or SEQ ID NO: 14. In one aspect, the polypeptides differ by up to 10 amino acids, e.g., 1, 2, 3, 4, 5, 6, 7, 8, 9, or 10, from the polypeptide comprising SEQ ID NO: 1 or SEQ ID NO: 14.

In an embodiment, the present invention relates to a DNase obtained from Bacillus in particular Bacillus licheniformis. In an embodiment, the present invention relates to a DNase polypeptide comprising the amino acid sequence of SEQ ID NO: 2 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 2. In one aspect, the polypeptides differ by up to 10 amino acids, e.g., 1, 2, 3, 4, 5, 6, 7, 8, 9, or 10, from the polypeptide comprising SEQ ID NO: 2.

In an embodiment, the present invention relates to a DNase obtained from Bacillus in particular Bacillus subtilis. In an embodiment, the present invention relates to a DNase polypeptide comprising the amino acid sequence of SEQ ID NO: 3 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 3. In one aspect, the polypeptides differ by up to 10 amino acids, e.g., 1, 2, 3, 4, 5, 6, 7, 8, 9, or 10, from the polypeptide comprising SEQ ID NO: 3.

In an embodiment, the present invention relates to a DNase obtained from Serratia in particular Serratia marcescens. In an embodiment, the present invention relates to a DNase polypeptide comprising the amino acid sequence of SEQ ID NO: 4 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 4. In one aspect, the polypeptides differ by up to 10 amino acids, e.g., 1, 2, 3, 4, 5, 6, 7, 8, 9, or 10, from the polypeptide comprising SEQ ID NO: 4.

In an embodiment, the present invention relates to a DNase obtained from Bacillus in particular Bacillus idriensis. In an embodiment, the present invention relates to a DNase polypeptide comprising the amino acid sequence of SEQ ID NO: 5 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO 5. In one aspect, the polypeptides differ by up to 10 amino acids, e.g., 1, 2, 3, 4, 5, 6, 7, 8, 9, or 10, from the polypeptide comprising SEQ ID NO: 5.

In an embodiment, the present invention relates to a DNase obtained from Bacillus in particular Bacillus cibi. In an embodiment, the present invention relates to a DNase polypeptide comprising the amino acid sequence of SEQ ID NO: 6 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 6. In one aspect, the polypeptides differ by up to 10 amino acids, e.g., 1, 2, 3, 4, 5, 6, 7, 8, 9, or 10, from the polypeptide comprising SEQ ID NO: 6.

In an embodiment, the present invention relates to a DNase obtained from Bacillus in particular Bacillus horikoshii. In an embodiment, the present invention relates to a DNase polypeptide comprising the amino acid sequence of SEQ ID NO: 7 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 7. In one aspect, the polypeptides differ by up to 10 amino acids, e.g., 1, 2, 3, 4, 5, 6, 7, 8, 9, or 10, from the polypeptide comprising SEQ ID NO: 7.

In an embodiment, the present invention relates to a DNase obtained from Bacillus sp. In an embodiment, the present invention relates to a DNase polypeptide comprising the amino acid sequence of SEQ ID NO: 8 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 8. In one aspect, the polypeptides differ by up to 10 amino acids, e.g., 1, 2, 3, 4, 5, 6, 7, 8, 9, or 10, from the polypeptide comprising SEQ ID NO: 8.

In an embodiment, the present invention relates to a DNase obtained from Bacillus sp. In an embodiment, the present invention relates to a DNase polypeptide comprising the amino acid sequence of SEQ ID NO: 9 or comprising an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 9. In one aspect, the polypeptides differ by up to 10 amino acids, e.g., 1, 2, 3, 4, 5, 6, 7, 8, 9, or 10, from the polypeptide comprising SEQ ID NO: 9.

In another embodiment, the DNase of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 14 comprises a substitution, deletion, and/or insertion at one or more (e.g., several) positions. In another embodiment, the DNase of SEQ ID NO: 9 comprises a substitution, deletion, and/or insertion at one or more (e.g., several) positions. In an embodiment, the number of amino acid substitutions, deletions and/or insertions introduced into the polypeptide SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 14 is not more than 10, e.g., 1, 2, 3, 4, 5, 6, 7, 8 or 9. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for DNase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The concentration of the DNase in the wash liquor is typically in the range of 0.00004-100 ppm enzyme protein, such as in the range of 0.00008-100, in the range of 0.0001-100, in the range of 0.0002-100, in the range of 0.0004-100, in the range of 0.0008-100, in the range of 0.001-100 ppm enzyme protein, 0.01-100 ppm enzyme protein, preferably 0.05-50 ppm enzyme protein, more preferably 0.1-50 ppm enzyme protein, more preferably 0.1-30 ppm enzyme protein, more preferably 0.5-20 ppm enzyme protein, and most preferably 0.5-10 ppm enzyme protein.

The DNase of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Liquid Enzyme Formulations

The DNase may be formulated as a liquid enzyme formulation, which is generally a pourable composition, though it may also have a high viscosity. The physical appearance and properties of a liquid enzyme formulation may vary a lot—for example, they may have different viscosities (gel to water-like), be colored, not colored, clear, hazy, and even with solid particles like in slurries and suspensions. The minimum ingredients are the DNase and a solvent system to make it a liquid. In addition to the DNase, the liquid enzyme formulation may also comprise other enzyme activities, such as protease, amylase, lipase, cellulase, and/or additional nuclease (e.g., RNase) activities.

The solvent system may comprise water, polyols (such as glycerol, (mono, di, or tri) propylene glycol, (mono, di, or tri) ethylene glycol, sugar alcohol (e.g. sorbitol, mannitol, erythritol, dulcitol, inositol, xylitol or adonitol), polypropylene glycol, and/or polyethylene glycol), ethanol, sugars, and salts. Usually the solvent system also includes a preservation agent and/or other stabilizing agents.

A liquid enzyme formulation may be prepared by mixing a solvent system and an enzyme concentrate with a desired degree of purity (or enzyme particles to obtain a slurry/suspension).

In an embodiment, the liquid enzyme composition comprises:

(a) at least 0.01% w/w active enzyme protein,
(b) at least 0.5% w/w polyol,
(c) water, and
(d) optionally a preservation agent.

The DNase in the liquid composition of the invention may be stabilized using conventional stabilizing agents. Examples of stabilizing agents include, but are not limited to, sugars like glucose, fructose, sucrose, or trehalose; polyols like glycerol, propylene glycol; addition of salt to increase the ionic strength; divalent cations (e.g., Ca²⁺ or Mg²⁺); and enzyme inhibitors, enzyme substrates, or various polymers (e.g., PVP). Selecting the optimal pH for the formulation may be very important for enzyme stability. The optimal pH depends on the specific enzyme but is typically in the range of pH 4-9. In some cases, surfactants like nonionic surfactant (e.g., alcohol ethoxylates) can improve the physical stability of the enzyme formulations.

One embodiment of the invention relates to a composition comprising a DNase, wherein the composition further comprises:

(i) a polyol, preferably selected from glycerol, (mono, di, or tri) propylene glycol, (mono, di, or tri) ethylene glycol, polyethylene glycol, sugar alcohols, sorbitol, mannitol, erythritol, dulcitol, inositol, xylitol and adonitol;
(ii) optionally an additional enzyme, preferably selected from protease, amylase, or lipase;
(iii) optionally a surfactant, preferably selected from anionic and nonionic surfactants;
(iv) optionally a divalent cation, polymer, or enzyme inhibitor;
(v) optionally having a pH in the range of pH 4-9; and
(vi) water.

Slurries or dispersions of enzymes are typically prepared by dispersing small particles of enzymes (e.g., spray-dried particles) in a liquid medium in which the enzyme is sparingly soluble, e.g., a liquid nonionic surfactant or a liquid polyethylene glycol. Powder can also be added to aqueous systems in an amount so not all go into solution (above the solubility limit). Another format is crystal suspensions which can also be aqueous liquids (see for example WO2019/002356). Another way to prepare such dispersion is by preparing water-in-oil emulsions, where the enzyme is in the water phase, and evaporate the water from the droplets. Such slurries/suspension can be physically stabilized (to reduce or avoid sedimentation) by addition of rheology modifiers, such as fumed silica or xanthan gum, typically to get a shear thinning rheology.

### Granular Enzyme Formulations

The DNase may also be formulated as a solid/granular enzyme formulation. Non-dusting granulates may be produced, e.g. as disclosed in U.S. Pat. Nos. 4,106,991 and 4,661,452, and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The DNase may be formulated as a granule for example as a co-granule that combines one or more enzymes or benefit agents (such as MnTACN or other bleaching components). Examples of such additional enzymes include proteases, amylases, lipases, cellulases, and/or nucleases (e.g., DNase, RNase). Each enzyme will then be present in more granules securing a more uniform distribution of enzymes in the detergent. This also reduces the physical segregation of different enzymes due to different particle sizes. Methods for producing multi-enzyme co-granulate for the detergent industry are disclosed in the IP.com disclosure IPCOM000200739D.

An embodiment of the invention relates to an enzyme granule/particle comprising a DNase. The granule is composed of a core, and optionally one or more coatings (outer layers) surrounding the core. Typically, the granule/particle size, measured as equivalent spherical diameter (volume based average particle size), of the granule is 20-2000 μm, particularly 50-1500 μm, 100-1500 μm or 250-1200 μm.

The core may include additional materials such as fillers, fibre materials (cellulose or synthetic fibers), stabilizing agents, solubilising agents, suspension agents, viscosity regulating agents, light spheres, plasticizers, salts, lubricants and fragrances. The core may include binders, such as synthetic polymer, wax, fat, or carbohydrate. The core may comprise a salt of a multivalent cation, a reducing agent, an antioxidant, a peroxide decomposing catalyst and/or an acidic buffer component, typically as a homogenous blend. The core may consist of an inert particle with the enzyme absorbed into it, or applied onto the surface, e.g., by fluid bed coating. The core may have a diameter of 20-2000 μm, particularly 50-1500 μm, 100-1500 μm or 250-1200 μm. The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation. Methods for preparing the core can be found in Handbook of Powder Technology; Particle size enlargement by C. E. Capes; Volume 1; 1980; Elsevier. These methods are well-known in the art and have also been described in international patent application WO2015/028567, pages 3-5, which is incorporated by reference.

The core of the enzyme granule/particle may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA). Examples of enzyme granules with multiple coatings are shown in WO 93/07263 and WO 97/23606.

Such coatings are well-known in the art, and have earlier been described in, for example, WO00/01793, WO2001/025412, and WO2015/028567, which are incorporated by reference.

In one aspect, the present invention provides a granule, which comprises:

(a) a core comprising a DNase according to the invention; and
(b) optionally a (salt) coating consisting of one or more layer(s) surrounding the core.

Another aspect of the invention relates to a layered granule, comprising:

(a) a (non-enzymatic) core;
(b) a coating surrounding the core, wherein the coating comprises a DNase; and
(c) optionally a (salt) coating consisting of one or more layer(s) surrounding the enzyme containing coating.

### Encapsulated Enzyme Formulations

The DNase may also be formulated as an encapsulated enzyme formulation (an ‘encapsulate’). This is particularly useful for separating the enzyme from other ingredients when the enzyme is added into, for example, a (liquid) cleaning composition, such as the detergent compositions described below.

Physical separation can be used to solve incompatibility between the enzyme(s) and other components. Incompatibility can arise if the other components are either reactive against the enzyme, or if the other components are substrates of the enzyme. Other enzymes can be substrates of proteases.

The enzyme may be encapsulated in a matrix, preferably a water-soluble or water dispersible matrix (e.g., water-soluble polymer particles), for example as described in WO 2016/023685. An example of a water-soluble polymeric matrix is a matrix composition comprising polyvinyl alcohol. Such compositions are also used for encapsulating detergent compositions in unit-dose formats.

The enzyme may also be encapsulated in core-shell microcapsules, for example as described in WO 2015/144784, or as described in the IP.com disclosure IPCOM000239419D.

Such core-shell capsules can be prepared using a number of technologies known in the art, e.g., by interfacial polymerization using either a water-in-oil or an oil-in-water emulsion, where polymers are crosslinked at the surface of the droplets in the emulsion (the interface between water and oil), thus forming a wall/membrane around each droplet/capsule.

### Formulation of Enzyme in Co-Granule

The DNase may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise a DNase and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### Purity of Enzyme in Formulations

The DNase used in the above-mentioned enzyme formulations may be purified to any desired degree of purity. This includes high levels of purification, as achieved for example by using methods of crystallization—but also none or low levels of purification, as achieved for example by using crude fermentation broth, as described in WO 2001/025411, or in WO 2009/152176.

### Microorganisms

The enzyme formulations, as well as the detergent formulations described below, may comprise one or more microorganisms or microbes. Generally, any microorganism(s) may be used in the enzyme/detergent formulations in any suitable amount(s)/concentration(s). Microorganisms may be used as the only biologically active ingredient, but they may also be used in conjunction with one or more of the enzymes described above.

The purpose of adding the microorganism(s) may, for example, be to reduce malodor as described in WO 2012/112718. Other purposes could include in-situ production of desirable biological compounds, or inoculation/population of a locus with the microorganism(s) to competitively prevent other non-desirable microorganisms form populating the same locus (competitive exclusion).

The term “microorganism” generally means small organisms that are visible through a microscope. Microorganisms often exist as single cells or as colonies of cells. Some microorganisms may be multicellular. Microorganisms include prokaryotic (e.g., bacteria and archaea) and eurkaryotic (e.g., some fungi, algae, protozoa) organisms. Examples of bacteria may be Gram-positive bacteria or Gram-negative bacteria. Example forms of bacteria include vegetative cells and endospores. Examples of fungi may be yeasts, molds and mushrooms. Example forms of fungi include hyphae and spores. Herein, viruses may be considered microorganisms.

Microorganisms may be recombinant or non-recombinant. In some examples, the microorganisms may produce various substances (e.g., enzymes) that are useful for inclusion in detergent compositions. Extracts from microorganisms or fractions from the extracts may be used in the detergents. Media in which microorganisms are cultivated or extracts or fractions from the media may also be used in detergents. In some examples, specific of the microorganisms, substances produced by the microorganisms, extracts, media, and fractions thereof, may be specifically excluded from the detergents. In some examples, the microorganisms, or substances produced by, or extracted from, the microorganisms, may activate, enhance, preserve, prolong, and the like, detergent activity or components contained with detergents.

Generally, microorganisms may be cultivated using methods known in the art. The microorganisms may then be processed or formulated in various ways. In some examples, the microorganisms may be desiccated (e.g., lyophilized). In some examples, the microorganisms may be encapsulated (e.g., spray drying). Many other treatments or formulations are possible. These treatments or preparations may facilitate retention of microorganism viability over time and/or in the presence of detergent components. In some examples, however, microorganisms in detergents may not be viable. The processed/formulated microorganisms may be added to detergents prior to, or at the time the detergents are used.

In one embodiment, the microorganism is a species of Bacillus, for example, at least one species of Bacillus selected from the group consisting of Bacillus subtilis, Bacillus amyloliquefaciens, Bacillus licheniformis, Bacillus atrophaeus, Bacillus pumilus, Bacillus megaterium, or a combination thereof. In a preferred embodiment, the aforementioned Bacillus species are on an endospore form, which significantly improves the storage stability.

### Detergent Compositions

In one embodiment, the invention is directed to detergent compositions comprising an enzyme of the present invention in combination with one or more additional cleaning composition components. In one embodiment, the detergent composition comprises a polypeptide having DNase activity comprising the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. The detergent composition may additionally comprise a cellulase having an amino acid sequence set forth in SEQ ID NO: 10, SEQ ID NO: 11, SEQ ID NO: 12, SEQ ID NO: 13, or SEQ ID NO: 14. In one embodiment the detergent composition is in solid form. In another embodiment, the detergent composition is in a liquid or gel form. In another embodiment a bar form. In one embodiment the detergent may be wrapped in water soluble PVOH film. The choice of additional components is within the skill of the artisan and includes conventional ingredients, including the exemplary non-limiting components set forth below.

### Liquid Detergent Composition

The liquid detergent composition may comprise a microcapsule of the invention, and thus form part of, any detergent composition in any form, such as liquid and powder detergents, and soap and detergent bars.

In one embodiment, the invention is directed to liquid detergent compositions comprising a microcapsule, as described above, in combination with one or more additional cleaning composition components.

The microcapsule, as described above, may be added to the liquid detergent composition in an amount corresponding to from 0.0001% to 5% (w/w) active enzyme protein (AEP); preferably from 0.001% to 5%, more preferably from 0.005% to 5%, more preferably from 0.005% to 4%, more preferably from 0.005% to 3%, more preferably from 0.005% to 2%, even more preferably from 0.01% to 2%, and most preferably from 0.01% to 1% (w/w) active enzyme protein.

The liquid detergent composition has a physical form, which is not solid (or gas). It may be a pourable liquid, a paste, a pourable gel or a non-pourable gel. It may be either isotropic or structured, preferably isotropic. It may be a formulation useful for washing in automatic washing machines or for hand washing. It may also be a personal care product, such as a shampoo, toothpaste, or a hand soap.

The liquid detergent composition may be aqueous, typically containing at least 20% by weight and up to 95% water, such as up to 70% water, up to 50% water, up to 40% water, up to 30% water, or up to 20% water. Other types of liquids, including without limitation, alkanols, amines, diols, ethers and polyols may be included in an aqueous liquid detergent. An aqueous liquid detergent may contain from 0-30% organic solvent. A liquid detergent may even be non-aqueous, wherein the water content is below 10%, preferably below 5%.

Detergent ingredients can be separated physically from each other by compartments in water dissolvable pouches. Thereby negative storage interaction between components can be avoided. Different dissolution profiles of each of the compartments can also give rise to delayed dissolution of selected components in the wash solution.

The detergent composition may take the form of a unit dose product. A unit dose product is the packaging of a single dose in a non-reusable container. It is increasingly used in detergents for laundry. A detergent unit dose product is the packaging (e.g., in a pouch made from a water-soluble film) of the amount of detergent used for a single wash.

Pouches can be of any form, shape and material which is suitable for holding the composition, e.g., without allowing the release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, maltodextrin, polymethacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be a blend composition comprising hydrolytically degradable and water soluble polymer blends such as polyactide and polyvinyl alcohol (known under the Trade reference M8630 as sold by Chris Craft In. Prod. Of Gary, Ind., US) plus plasticizers like glycerol, ethylene glycerol, Propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids (see e.g., US 2009/0011970).

The choice of detergent components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The choice of additional components is within the skill of the artisan and includes conventional ingredients, including the exemplary non-limiting components set forth below.

### Surfactants

The cleaning composition may comprise one or more surfactants, which may be anionic and/or cationic and/or non-ionic and/or semi-polar and/or zwitterionic, or a mixture thereof. In a particular embodiment, the detergent composition includes a surfactant system (comprising more than one surfactant) e.g. a mixture of one or more nonionic surfactants and one or more anionic surfactants. In one embodiment the detergent comprises at least one anionic surfactant than at least one non-ionic surfactant, the weight ratio of anionic to nonionic surfactant may be from 10:1 to 1:10. In one embodiment the amount of anionic surfactant is higher than the amount of non-ionic surfactant e.g. the weight ratio of anionic to non-ionic surfactant may be from 10:1 to 1.1:1 or from 5:1 to 1.5:1. The amount of anionic to non-ionic surfactant may also be equal and the weight ratios 1:1. In one embodiment the amount of non-ionic surfactant is higher than the amount of anionic surfactant and the weight ratio may be 1:10 to 1:1.1. Preferably the weight ratio of anionic to non-ionic surfactant is from 10:1 to 1:10, such as from 5:1 to 1:5, or from 5:1 to 1:1.2. Preferably, the weight fraction of non-ionic surfactant to anionic surfactant is from 0 to 0.5 or 0 to 0.2 thus non-ionic surfactant can be present or absent if the weight fraction is 0, but if non-ionic surfactant is present, then the weight fraction of the nonionic surfactant is preferably at most 50% or at most 20% of the total weight of anionic surfactant and non-ionic surfactant. Light duty detergent usually comprises more nonionic than anionic surfactant and there the fraction of non-ionic surfactant to anionic surfactant is preferably from 0.5 to 0.9. The total weight of surfactant(s) is typically present at a level of from about 0.1% to about 60% by weight, such as about 1% to about 40%, or about 3% to about 20%, or about 3% to about 10%. The surfactant(s) is chosen based on the desired cleaning application, and may include any conventional surfactant(s) known in the art. When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, typically available as sodium or potassium salts or salts of monoethanolamine (MEA, 2-aminoethan-1-ol) or triethanolamine (TEA, 2,2′,2″-nitrilotriethan-1-ol); in particular, linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids. Furthermore, salts of fatty acids (soaps) may be included.

When included therein the detergent will usually contain from about 1% to about 40% by weight of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO) e.g. the AEO-series such as AEO-7, alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.01 to about 10% by weight of a semipolar surfactant. Non-limiting examples of semipolar surfactants include amine oxides (AO) such as alkyldimethylamine oxides, in particular N-(coco alkyl)-N,N-dimethylamine oxide and N-(tallow-alkyl)-N,N-bis(2-hydroxyethyl)amine oxide, and combinations thereof.

When included therein the detergent will usually contain from about 0.01% to about 10% by weight of a zwitterionic surfactant. Non-limiting examples of zwitterionic surfactants include betaines such as alkyldimethylbetaines, sulfobetaines, and combinations thereof.

Additional bio-based surfactants may be used e.g. wherein the surfactant is a sugar-based non-ionic surfactant which may be a hexyl-β-D-maltopyranoside, thiomaltopyranoside or a cyclic-maltopyranoside, such as described in EP2516606 B1.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically in the range 40-65%, particularly in the range 50-65%. The builder and/or co-builder may particularly be a chelating agent that forms water-soluble complexes with Ca and Mg. Any builder and/or co-builder known in the art for use in cleaning detergents may be utilized.

Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Clariant), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain from about 0-50% by weight, such as about 5% to about 30%, of a detergent co-builder. The detergent composition may include a co-builder alone, or in combination with a builder, for example a zeolite builder. Non-limiting examples of co-builders include or copolymers thereof, such as poly(acrylic acid) (PAA) or copoly(acrylic acid/maleic acid) (PAA/PMA). According to the present invention, these components can be included in lower levels than in currently available detergent compositions. Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid (HEDP), ethylenediaminetetramethylenetetrakis(phosphonic acid) (EDTMPA), diethylenetriaminepentamethylenepentakis(phosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)aspartic acid (SMAS), N-(2-sulfoethyl)aspartic acid (SEAS), N-(2-sulfomethyl)glutamic acid (SMGL), N-(2-sulfoethyl)glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), aminotrimethylenetris(phosphonic acid) (ATMP), and combinations and salts thereof. Further exemplary builders and/or co-builders are described in, e.g., WO 09/102854, U.S. Pat. No. 5,977,053.

### Bleaching Systems

The cleaning composition may contain 0-50% by weight, such as 1-40%, such as 1-30%, such as about 1% to about 20%, of a bleaching system. Any oxygen-based bleaching system comprising components known in the art for use in cleaning detergents may be utilized. Suitable bleaching system components include sources of hydrogen peroxide; peracids and sources of peracids (bleach activators); and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds, such as iron or cobalt complexes.

In some embodiments, where a source of a peracid is included, an organic bleach catalyst or bleach booster may be used having one of the following formulae:

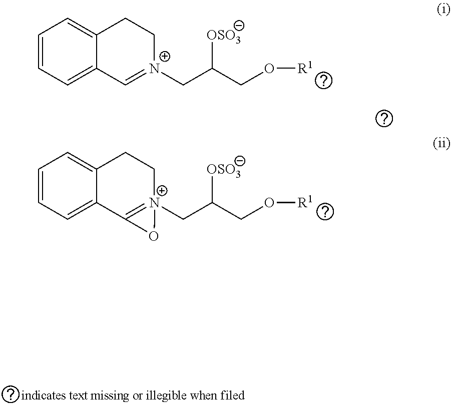

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

### Polymers

Generally, detergent compositions may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include polyethylene oxide and polypropylene oxide (PEO-PPO), diquaternium ethoxy sulfate, styrene/acrylic copolymer and perfume capsules Other exemplary polymers are disclosed in, e.g., WO 2006/130575. Salts of the above-mentioned polymers are also contemplated.

According to the present invention, however, certain of the above polymers, namely, a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, can be included in lower levels than in currently available detergent compositions, or even more preferably, excluded altogether in that it can be replaced or partly replaced by DNase.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Additional Enzymes

The detergent additive as well as the detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

In an embodiment, the cellulase is obtained from Humicola in particular Humicola insolens. In an embodiment, cellulase comprises the amino acid sequence of SEQ ID NO: 10 or comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO 10. In one aspect, the polypeptides differ by up to 10 amino acids, e.g., 1, 2, 3, 4, 5, 6, 7, 8, 9, or 10, from the polypeptide comprising SEQ ID NO: 10.

In an embodiment, the cellulase is obtained from Bacillus in particular Bacillus akibai. In an embodiment, the cellulase comprises the amino acid sequence of SEQ ID NO: 11 or comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO 11. In one aspect, the polypeptides differ by up to 10 amino acids, e.g., 1, 2, 3, 4, 5, 6, 7, 8, 9, or 10, from the polypeptide comprising SEQ ID NO: 11.

In an embodiment, the cellulase is obtained from Paenibacillus in particular Paenibacillus polymyxa. In an embodiment, the cellulase comprises the amino acid sequence of SEQ ID NO: 12 or comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO:12. In one aspect, the polypeptides differ by up to 10 amino acids, e.g., 1, 2, 3, 4, 5, 6, 7, 8, 9, or 10, from the polypeptide comprising SEQ ID NO: 12.

In an embodiment, the cellulase is obtained from Melanocarpus in particular Melanocarpus albomyces. In an embodiment, the cellulase comprises the amino acid sequence of SEQ ID NO: 13 or comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO:13. In one aspect, the polypeptides differ by up to 10 amino acids, e.g., 1, 2, 3, 4, 5, 6, 7, 8, 9, or 10, from the polypeptide comprising SEQ ID NO: 13.

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Proteases

Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Examples of preferred proteases include:

(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V205I, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;

(b) a variant of the polypeptide of SEQ ID NO: 1 of WO 2016/001449 with the mutation S99SE, wherein position numbers are based on the numbering of SEQ ID NO: 2 of WO 2016/001449;

(c) a variant of the polypeptide of SEQ ID NO: 1 of WO 2016/001449 with the mutation S99AD, wherein position numbers are based on the numbering of SEQ ID NO: 2 of WO 2016/001449;

(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;

(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;

(f) a variant of the polypeptide of SEQ ID NO: 1 of WO 2016/001449 with the substitutions S9R+A15T+G61E+V68A+A194P+V205I+Q245R+N261D, wherein position numbers are based on the numbering of SEQ ID NO: 2 of WO 2016/001449;

(g) a variant of the polypeptide of SEQ ID NO: 1 of WO 2016/001449 with the substitutions S99D+S101R/E+S103A+V104I+G160S; for example a variant of SEQ ID NO: 1 of WO 2016/001449 with the substitutions S3T+V4I+S99D+S101E+S103A+V104I+G160S+V205I, wherein position numbers are based on the numbering of SEQ ID NO: 2 of WO 2016/001449;

(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;

(i) the polypeptide disclosed in GENESEQP under accession number BER84782, corresponding to SEQ ID NO: 302 in WO 2017/210295;

(j) a variant of the polypeptide of SEQ ID NO: 1 of WO 2016/001449 with the substitutions S99D+S101E+S103A+V104I+S156D+G160S+L262E, wherein position numbers are based on the numbering of SEQ ID NO: 2 of WO 2016/001449;

(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;

(l) a variant of the polypeptide of SEQ ID NO: 1 of WO 2016/001449 with the substitutions V68A+S106A, wherein position numbers are based on the numbering of SEQ ID NO: 2 of WO 2016/001449; and

(m) a variant of the polypeptide of SEQ ID NO: 1 of WO 2004/067737 with the substitutions S27K+N109K+S111E+5171E+S173P+G174K+S175P+F180Y+G182A+L184F+Q198E+N199+T297P, wherein position numbers are based on the numbering of SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Purafect Prime, Preferenz P110™, Effectenz P1000™, Purafect®, Effectenz P1050™, Purafect® Ox, Effectenz™ P2000, Purafast™ Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases include an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme PIus™, Natalase™, Liquozyme X and BAN™ (from Novozymes A/S), and Rapidase™ Purastar™/Effectenz™, Powerase, Preferenz S1000, Preferenz S100 and Preferenz S110 (from Genencor International Inc./DuPont).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme (Novozymes A/S).

A suitable peroxidase is preferably a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus. A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Other Materials

Any detergent components known in the art for use in detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

According to the present invention, however, certain of the above polymers, namely, a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, can be included in lower levels than in currently available detergent compositions, or even more preferably, excluded altogether.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Tinopal CBS-X is a 4,4′-bis-(sulfostyryl)-biphenyl disodium salt also known as Disodium Distyrylbiphenyl Disulfonate. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference).

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

According to the present invention, however, certain of the above polymers, namely, a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, can be included in lower levels than in currently available detergent compositions, or even more preferably, excluded altogether in that it can be replaced or partly replaced by DNase.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The DNase of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, DNase, optionally one or more additional enzymes, a protease inhibitor, and a salt of a monovalent cation and an organic anion may be prepared and and the mixture is then plodded. The DNase and optional additional enzymes may be added at the same time as the protease inhibitor for example in liquid form. Besides the mixing step and the plodding step, the process may further comprise the steps of milling, extruding, cutting, stamping, cooling and/or wrapping.

The invention is further summarized in the following paragraphs:

Paragraph 1. Use in detergent of a polypeptide having DNase activity for maintaining or improving whiteness of an item during a wash cycle in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, wherein the item is a textile.
Paragraph 2. Use in detergent of a polypeptide having DNase activity for maintaining or improving whiteness of an item during a wash cycle as a reduction or even replacement for a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.
Paragraph 3. Use in detergent of a polypeptide having DNase activity for preventing, reducing or removing redeposition of soil to an item during a wash cycle in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, wherein the item is a textile.
Paragraph 4. Use of any preceding paragraph, wherein the polypeptide having DNase activity is obtained from a fungal source.
Paragraph 5. Use of any preceding paragraph, wherein the polypeptide having DNase activity is obtained from Aspergillus, e.g., A. oryzae.
Paragraph 6. Use of any preceding paragraph, wherein the polypeptide having DNase activity is obtained from a bacterial source.
Paragraph 7. Use of any preceding paragraph, wherein the polypeptide having DNase activity is obtained from Bacillus, e.g. B. cibi.
Paragraph 8. Use of any preceding paragraph, wherein the polypeptide having DNase activity has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14 or a polypeptide having at least 60%, at least 65%, at least 70%, at least 75%, at least 80%, at least 85%, at least 90%, at least 95%, or even 100% sequence identity thereto.
Paragraph 9. Use of any preceding paragraph, which provides improved whiteness and/or improved antiredeposition compared to use in the presence of the polyacrylic acid, modified polyacrylic acid polymer, modified polyacrylic acid copolymer, maleic acid-acrylic acid copolymer, carboxymethyl cellulose, cellulose gum, methyl cellulose, and/or combinations thereof.
Paragraph 10. Use of any preceding paragraph, wherein the detergent is a liquid detergent in the absence of a a polyacrylic acid, a modified polyacrylic acid polymer, a modified polyacrylic acid copolymer, a maleic acid-acrylic acid copolymer, and/or combinations thereof.
Paragraph 11. Use of any of paragraphs 1-9, wherein the detergent is a powder detergent in the absence of a carboxymethyl cellulose, cellulose gum, methyl cellulose, and combinations thereof.
Paragraph 12. Use of any preceding paragraph, further comprising use of a polypeptide having cellulase activity.
Paragraph 13. Use of paragraph 12, wherein the polypeptide having cellulase activity has an amino acid sequence selected from the group consisting of SEQ ID NO: 10, SEQ ID NO: 11, SEQ ID NO: 12, SEQ ID NO: 13 or a polypeptide having at least 60%, at least 65%, at least 70%, at least 75%, at least 80%, at least 85%, at least 90%, at least 95%, or even 100% sequence identity thereto.
Paragraph 14. A detergent composition comprising a polypeptide having deoxyribonuclease (DNase) activity and a detergent adjunct ingredient, provided that the composition comprises less than 1%, e.g., less than 0.8%, less than 0.7%, less than 0.6%, less than 0.5%, less than 0.4%, less than 0.3%, less than 0.2%, less than 0.1%, less than 0.05%, less than 0,025% by weight of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.
Paragraph 15. Detergent composition of any preceding composition paragraph, wherein the polypeptide is obtained from a fungal source.
Paragraph 16. Detergent composition of any preceding composition paragraph, wherein the polypeptide is obtained from Aspergillus, e.g., A. oryzae.
Paragraph 17. Detergent composition of any preceding composition paragraph, wherein the polypeptide is obtained from a bacterial source.
Paragraph 18. Detergent composition of any preceding composition paragraph, wherein the polypeptide is obtained from Bacillus, e.g., B. cibi.
Paragraph 19. Detergent composition of any preceding composition paragraph, wherein the polypeptide has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14 or a polypeptide having at least 60%, at least 65%, at least 70%, at least 75%, at least 80%, at least 85%, at least 90%, at least 95%, or even 100% sequence identity thereto.
Paragraph 20. Detergent composition of any preceding composition paragraph, which is a liquid detergent.
Paragraph 21. Detergent composition of any of paragraphs 14-19 which is a powder detergent.
Paragraph 22. Detergent composition of any preceding detergent composition paragraph, further comprising a polypeptide having cellulase activity.
Paragraph 23. Detergent composition of paragraph 22, wherein the polypeptide having cellulase activity has an amino acid sequence selected from the group consisting of SEQ ID NO: 10, SEQ ID NO: 11, SEQ ID NO: 12, SEQ ID NO: 13 or a polypeptide having at least 60%, at least 65%, at least 70%, at least 75%, at least 80%, at least 85%, at least 90%, at least 95%, or even 100% sequence identity thereto.
Paragraph 24. A method for laundering an item, which method comprises the steps of:
a) exposing an item to a wash liquor comprising a polypeptide having DNase activity or a detergent composition comprising the polypeptide in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof;
b) completing at least one wash cycle;
c) optionally adding additional soiling; and
d) optionally rinsing the item, wherein the item is a textile.
   Paragraph 25. The method of paragraph 24, wherein the polypeptide having DNase activity provides the same or better whiteness of the item compared to a laundering method performed with a detergent composition with the polyacrylic acid, modified polyacrylic acid polymer, modified polyacrylic acid copolymer, maleic acid-acrylic acid copolymer, carboxymethyl cellulose, cellulose gum, methyl cellulose, and/or combinations thereof.
   Paragraph 26. Method of any preceding method paragraph, wherein the polypeptide is obtained from a fungal source.
   Paragraph 27. Method of any preceding method paragraph, wherein the polypeptide is obtained from Aspergillus, e.g., A. oryzae.
   Paragraph 28. Method of any preceding method paragraph, wherein the polypeptide is obtained from a bacterial source.
   Paragraph 29. Method of any preceding method paragraph, wherein the polypeptide is obtained from Bacillus, e.g. B. cibi.
   Paragraph 30. Method of any preceding method paragraph, wherein the polypeptide has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14 or a polypeptide having at least 60%, at least 65%, at least 70%, at least 75%, at least 80%, at least 85%, at least 90%, at least 95%, or even 100% sequence identity thereto.
   Paragraph 31. Method of any preceding method paragraph, which provides improved whiteness and/or improved antiredeposition compared to the method in the presence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.
   Paragraph 32. Method of any preceding method paragraph, wherein the detergent is a liquid in the absence of a polyacrylic acid, a modified polyacrylic acid polymer, a modified polyacrylic acid copolymer, a maleic acid-acrylic acid copolymer, and/or combinations thereof.
   Paragraph 33. Method of any of paragraphs 24-31, wherein the detergent is a powder detergent in the absence of a carboxymethyl cellulose, cellulose gum, methyl cellulose, and/or combinations thereof.
   Paragraph 34. Method of any preceding method paragraph, further comprising a polypeptide having cellulase activity.
   Paragraph 35. Method of paragraph 34, wherein the polypeptide having cellulase activity has an amino acid sequence selected from the group consisting of SEQ ID NO: 10, SEQ ID NO: 11, SEQ ID NO: 12, SEQ ID NO: 13 or a polypeptide having at least 60%, at least 65%, at least 70%, at least 75%, at least 80%, at least 85%, at least 90%, at least 95%, or even 100% sequence identity thereto.

### Assays and Detergent Compositions

%. (all percentages are w/w active)
Model Powder Detergent—Model X with CMC
Ingredients: LAS ((C10-C13) alkylbenzene-sulfonic acid, sodium salt) 15%,
nonionic (AEO) (C12-C14 alcohol ethoxylate with an average of 7 EO) 2%,
soda ash (sodium carbonate), 20.1%,
hydrous sodium silicate (“disilicate”) 9.9%,
zeolite 4A 12.1%,
copoly(acrylic acid/maleic acid), sodium salt 1.3%,
sodium sulfate, 31.4%,

### CMC 2%.

Model Powder Detergent—Model X without CMC
Ingredients: LAS ((C10-C13) alkylbenzene-sulfonic acid, sodium salt) 15%,
nonionic (AEO) (C12-C14 alcohol ethoxylate with an average of 7 EO) 2%,
soda ash (sodium carbonate), 20.1%,
hydrous sodium silicate (“disilicate”) 9.9%,
zeolite 4A 12.1%,
copoly(acrylic acid/maleic acid), sodium salt 1.3%,
sodium sulfate, 31.4%.
Model Detergent A (Liquid)—with Polymer (Comparative)
Ingredients: 12% LAS (C10-C13) alkylbenzene-sulfonic acid, sodium salt), 11% nonionic (AEO) (C12-C14 alcohol ethoxylate with an average of 7 EO, 7% AEOS (SLES) with an average of 3 EO, 6% MPG (monopropylene glycol), 3% ethanol, 3% TEA, 2.75% coco fatty acid, 2.75% soy fatty acid, 2% glycerol, 2% sodium hydroxide, 2% sodium citrate, 1% sodium formate, 0.2% DTMPA and 0.2% polycarboxylic acid (Sokalan CP5). Water ad 100%.
Model Detergent A without Polymer
Ingredients: 12% LAS (C10-C13) alkylbenzene-sulfonic acid, sodium salt), 11% nonionic (AEO) (C12-C14 alcohol ethoxylate with an average of 7 EO, 7% AEOS (SLES) with an average of 3 EO, 6% MPG (monopropylene glycol), 3% ethanol, 3% TEA, 2.75% coco fatty acid, 2.75% soy fatty acid, 2% glycerol, 2% sodium hydroxide, 2% sodium citrate, 1% sodium formate, and 0.2% DTMPA. Water ad 100%.

### Detergent Compositions

The below mentioned ranges of detergent components are generally useful in the context of the low-polymer detergent compositions of the invention.

### Tables:

### Composition 1: Liquid Detergent

| Ingredient | Amount (in wt %) |
|---|---|
| Anionic detersive surfactant (such as alkyl benzene sulphonate, alkyl | from 0 wt % to 40 wt % |
| ether sulphate, alpha-olefin sulphonate, methyl ester sulphonate and | |
| mixtures) | |
| Non-ionic detersive surfactant (such as alkyl ethoxylated alcohol, | from 0 wt % to 40 wt % |
| alkylpoly glucosides; Glycereth-6 Laurate, biosurfactants, and mixtures) | |
| Other detersive surfactant (such as zwitterionic detersive surfactants, | from 0 wt % to 4 wt % |
| amphoteric surfactants, quaternary ammonium compounds and mixtures | |
| thereof) | |
| Carboxylate polymer (such as co-polymers of maleic acid and acrylic | from 0 wt % to 4 wt % |
| acid, add other PCA polymers, eg. Sokalan CP types, Acusol types, etc) | |
| Polyethylene glycol polymer (such as a polyethylene glycol polymer | from 0 wt % to 4 wt % |
| comprising poly vinyl acetate side chains, PEG/vinyl acetate co-polymer | |
| Eg Sokalan HP22 type) | |
| Polyester or terephthalate soil release polymer (such as | From 0 to 4 wt % |
| Polypropylene/Polyethylene Terephthalate; Polyethylene Terephthalate; | |
| Sulfonated Polyethylene/Polyethylene Terephthalate anionic polyester, | |
| nonionic polymer,examples are the REPEL-O-TEX ® line of polymers | |
| (Solvay), including REPEL-O-TEX ® Crystal, REPEL-O-TEX ® SRP-6 and | |
| REPEL-O-TEX ® SF-2, Marloquest ® polymers, such as Marloquest ® SL | |
| (Sasol),and/or TexCare ® polymers, including TexCare ® SRA-300, | |
| TexCare ®, TexCare ® SRN-170, TexCare ® SRN-240, TexCare ® SRN- | |
| 260, and TexCare ® SRN-325, (Clariant). | |
| Other polymer (such as amine polymers, dye PVP-NO/Polyvinyl | from 0 wt % to 10 wt % |
| Pyrrolidone N-oxide; Vinylpyrrolidone/vinylimidazole co-polymers, | |
| hexamethylenediamine derivative polymers, Ethoxylated polyethylene- | |
| polyamine; AZIRIDIN, HOMOPOLYMER, and mixtures thereof, eg. | |
| Sokalan HP types, Sokalan K types | |
| Other builder (such as sodium citrate and/or citric acid, ethanolamine | from 0 wt % to 10 wt % |
| (such as MEA, DEA and TEA) | |
| Carbonate salt (such as sodium carbonate and/or sodium bicarbonate) | from 0 wt % to 10 wt % |
| Solvents (such as, 1,2-propanediol, glycerol and ethanol) | 0 wt % to 40 wt % |
| Chelant (such as the phosphonates and aminocarboxylates | from 0 wt % to2 wt % |
| (ethylenediamine-N′N′-disuccinic acid (EDDS) and/or hydroxyethane | |
| diphosphonic acid (HEDP), diethylenetriamine penta(methylene | |
| phosphonic acid) (DTPMP), Diethylenetriamine-pentaacetic acid (DTPA), | |
| Ethylenediaminetetraacetic acid (EDTA), methylglycine diacetic acid | |
| (MGDA); glutamic acid-N,N-diacetic acid (GLDA)) | |
| Optical brightener (such as 4,4′-Distyryl biphenyl types, FWA 5; FWA 7; | from 0 wt % to 0.5 wt % |
| FWA 11 and the likes | |
| Formulated protease/s | from 0 wt % to 5 wt % |
| Formulated Amylase/s | from 0 wt % to 1 wt % |
| Formulated Cellulase/s | from 0 wt % to 1 wt % |
| Formulated Lipase/s | from 0 to 1 wt % |
| Other formulated enzyme (such as xyloglucanase, cutinase, pectate | from 0 wt % to 2 wt % |
| lyase, mannanase, bleaching enzyme) | |
| Formulated DNase/s | 0.000001-10% |
| Fabric softener (such as montmorillonite clay and/or polydimethylsiloxane | from 0 wt % to 4 wt % |
| (PDMS) | |
| Suds suppressor (such as silicone and/or fatty acid) | from 0 wt % to 10wt % |
| Perfume (such as perfume microcapsule, perfume extract, liquid perfume, | from 0 wt % to 1 wt % |
| and any combination thereof) | |
| Aesthetics (such as opacifiers and colorants) | from 0 wt % to 1 wt % |
| Preservatives (eg isothiazolinones, phenoxyethanol, etc) | From 0 wt % to 2 wt % |
| others | optional |
| Filler (such as water) | balance |

### Composition 2: Unit Dose

| Ingredient | Amount (in wt %) |
|---|---|
| Anionic detersive surfactant (such as alkyl benzene sulphonate, alkyl | from 0 wt % to 50 wt % |
| ether sulphate, alpha-olefin sulphonate, methyl ester sulphonate and | thereof |
| mixtures, as acids, neutralized salts or as monoethanolamine adducts) | |
| Non-ionic detersive surfactant (such as alkyl ethoxylated alcohol, alkylpoly | from 0 wt % to 50 wt % |
| glucosides; Glycereth-6 Laurate, biosurfactants and mixtures) | |
| Other detersive surfactant (such as zwitterionic detersive surfactants, | from 0 wt % to 5 wt % |
| amphoteric surfactants, quaternary ammonium compounds and mixtures | |
| thereof) | |
| Carboxylate polymer (such as co-polymers of maleic acid and acrylic acid, | from 0 wt % to 5 wt % |
| add other PCA polymers, eg. Sokalan CP types, Acusol types, etc) | |
| Polyethylene glycol polymer (such as a polyethylene glycol polymer | from 0 wt % to 5 wt % |
| comprising poly vinyl acetate side chains, PEG/vinyl acetate co-polymer | |
| Eg Sokalan HP22 type) | |
| Polyester or terephthalate soil release polymer (such as | From 0 to 5wt % |
| Polypropylene/Polyethylene Terephthalate; Polyethylene Terephthalate; | |
| Sulfonated Polyethylene/Polyethylene Terephthalate anionic polyester, | |
| nonionic polymer, examples are the REPEL-O-TEX ® line of polymers | |
| (Solvay), including REPEL-O-TEX ® Crystal, REPEL-O-TEX ® SRP-6 and | |
| REPEL-O-TEX ® SF-2, Marloquest ® polymers, such as Marloquest ® SL | |
| (Sasol), and/or TexCare ® polymers, including TexCare ® SRA-300, | |
| TexCare ®, TexCare ® SRN-170, TexCare ® SRN-240, and TexCare ® | |
| SRN-325, (Clariant). | |
| Other polymer (such as amine polymers, dye transfer inhibitor polymers, | from 0 wt % to 20 wt % |
| PVP-NO/Polyvinyl Pyrrolidone N-oxide; Vinylpyrrolidone/vinylimidazole | |
| co-polymers, hexamethylenediamine derivative polymers, Ethoxylated | |
| polyethylene-polyamine; AZIRIDIN, HOMOPOLYMER,and mixtures | |
| thereof, eg. Sokalan HP types, Sokalan K types | |
| Other builder (such as sodium citrate and/or citric acid, ethanolamine | from 0 wt % to 15 wt % |
| (such as MEA, DEA and TEA) | |
| Solvents (such as, 1,2-propanediol, 1,3-propanediol, glycerol, dipropylene | 10 wt % to 60 wt % |
| glycol, methylpropanediol, sorbitol and ethanol) | |
| Chelant (such as the phosphonates and aminocarboxylates | from 0 wt % to 4 wt % |
| (ethylenediamine-N′N′-disuccinic acid (EDDS) and/or hydroxyethane | |
| diphosphonic acid(HEDP), diethylenetriamine penta(methylene | |
| phosphonic acid) (DTPMP), Diethylenetriamine-pentaacetic acid (DTPA), | |
| Ethylenediaminetetraacetic acid (EDTA), methylglycine diacetic acid | |
| (MGDA); glutamic acid-N,N-diacetic acid (GLDA), as acids, neutralized | |
| salts or as monoethanolamine adducts) | |
| Optical brightener (such as 4,4′-Distyryl biphenyl types, FWA 5; FWA 7; | from 0 wt % to 2 wt % |
| FWA 11 and the likes) | |
| Formulated protease/s | from 0 wt % to 10 wt % |
| Formulated Amylase/s | from 0 wt % to 10 wt % |
| Formulated Cellulase/s | from 0 wt % to 5 wt % |
| Formulated Lipase/s | from 0 wt % to 5 wt % |
| Other formulated enzyme (such as xyloglucanase, cutinase, pectate | from 0 wt % to 5 wt % |
| lyase, mannanase, bleaching enzyme) | |
| Formulated DNase/s | 0.000001-10% |
| Fabric softener (such as montmorillonite clay and/or polydimethylsiloxane | from 0 wt % to 4 wt % |
| (PDMS) | |
| Suds suppressor (such as silicone and/or fatty acid (as acids, neutralized | from 0 wt % to 10 wt % |
| salts or as monoethanolamine adducts)) | |
| Perfume (such as perfume microcapsule, perfume extract, liquid perfume, | from 0 wt % to 5 wt % |
| and any combination thereof) | |
| Aesthetics (such as opacifiers and colorants) | from 0 wt % to 2 wt % |
| Preservatives (such as isothiazolinones, phenoxyethanol) | From 0 wt % to 2 wt % |
| water | From 2 wt % to 30 wt % |
| others | optional balance |
| Filler (such as solvents) | |

### Composition 3 Powder Detergent

| Ingredient | Amount (in wt %) |
|---|---|
| Anionic detersive surfactant (such as alkyl benzene sulphonate, alkyl ether | from 0 wt % to 30 wt % |
| sulphate, alpha-olefin sulphonate, methyl ester sulphonate and mixtures | thereof) |
| Non-ionic detersive surfactant (such as alkyl ethoxylated alcohol, alkylpoly | from 0 wt % to 10 wt % |
| glucosides; Glycereth-6 Laurate, biosurfactants, and mixtures) | |
| Other detersive surfactant (such as zwitterionic detersive surfactants, | from 0 wt % to 4 wt % |
| amphoteric surfactants, quaternary ammonium compounds and mixtures | |
| thereof) | |
| Carboxylate polymer (such as co-polymers of maleic acid and acrylic acid, | from 0 wt % to 4 wt % |
| add other PCA polymers, eg. Sokalan CP types, Acusol types, etc) | |
| Polyethylene glycol polymer (such as a polyethylene glycol polymer | from 0 wt % to 4 wt % |
| comprising poly vinyl acetate side chains, PEG/vinyl acetate co-polymer | |
| Eg Sokalan HP22 type) | |
| Polyester or terephthalate soil release polymer (such as | 0 to 2 wt % |
| Polypropylene/Polyethylene Terephthalate; Polyethylene Terephthalate; | |
| Sulfonated Polyethylene/Polyethylene Terephthalate anionic polyester, | |
| nonionic polymer, examples are the REPEL-O-TEX ® line of polymers | |
| (Solvay), including, REPEL-O-TEX ® SRP-6 and REPEL-O-TEX ® SF-2, | |
| Marloquest ® polymers, such as Marloquest ® SL (Sasol), and/or TexCare ® | |
| polymers like TexCare ® SRA 300 F (Clariant). | |
| Other polymer (such as amine polymers, dye PVP-NO/Polyvinyl | from 0 wt % to 10 wt % |
| Pyrrolidone N-oxide; Vinylpyrrolidone/vinylimidazole co-polymers, | |
| hexamethylenediamine derivative polymers, Ethoxylated polyethylene- | |
| polyamine; AZIRIDIN, HOMOPOLYMER, and mixtures thereof, eg. | |
| Sokalan HP types, Sokalan K types | |
| Cellulosic polymer (such as carboxymethyl cellulose, methyl cellulose and | from 0 wt % to 5 wt % |
| combinations thereof) | |
| Zeolite builder and phosphate builder (such as zeolite 4A and/or sodium | from 0 wt % to 50 wt % |
| tripolyphosphate) | |
| Other builder (such as sodium citrate and/or citric acid; Sodium tripoly | from 0 wt % to 20 wt % |
| phosphate (STPP)) | |
| Carbonate salt (such as sodium carbonate and/or sodium bicarbonate) | from 0 wt % to 50 wt % |
| Silicate salt (such as sodium silicate) | from 0 wt % to 15 wt % |
| Filler (such as sodium sulphate, sodium chloride and/or bio-fillers and/or | balance |
| water/solvents) | |
| Source of available oxygen (such as sodium percarbonate) | from 0 wt % to 30 wt % |
| Bleach activator (such as tetraacetylethylene diamine (TAED) and/or | from 0 wt % to 15 wt % |
| nonanoyloxybenzenesulphonate (NOBS) | |
| Bleach catalyst (such as oxaziridinium-based bleach catalyst and/or | from 0 wt % to 0.5 wt % |
| transition metal bleach catalyst) | |
| Other bleach (such as reducing bleach and/or pre- formed peracid) | from 0 wt % to 10 wt % |
| Chelant (such as the phosphonates and aminocarboxylates | from 0 wt % to2 wt % |
| (ethylenediamine-N′N′-disuccinic acid (EDDS) and/or hydroxyethane | |
| diphosphonic acid (HEDP), diethylenetriamine penta(methylene | |
| phosphonic acid) (DTPMP), Diethylenetriamine-pentaacetic acid (DTPA), | |
| Ethylenediaminetetraacetic acid (EDTA) , methylglycine diacetic acid | |
| (MGDA); glutamic acid-N,N-diacetic acid (GLDA)) | |
| Optical brightener (such as 4,4′-Distyryl biphenyl types, FWA 5; FWA 7; | from 0 wt % to 0.5 wt % |
| FWA 11 and the likes | |
| Photobleach (such as zinc and/or aluminium sulphonated phthalocyanine) | from 0 wt % to 0.5 wt % |
| Hueing agent (such as direct violet 99, acid red 52, acid blue 80, direct | from 0 wt % to 1 wt % |
| violet 9, solvent violet 13 and any combination thereof) | |
| Formulated protease/s | from 0 wt % to 5 wt % |
| Formulated Amylase/s | from 0 wt % to 1 wt % |
| Formulated Cellulase/s | from 0 wt % to 1 wt % |
| Formulated Lipase/s | from 0 to 1 wt % |
| Other formulated enzyme (such as xyloglucanase, cutinase, pectate lyase, | from 0 wt % to 2 wt % |
| mannanase, bleaching enzyme) | |
| Formulated DNase/s | 0.000001-10% |
| Fabric softener (such as montmorillonite clay and/or polydimethylsiloxane | from 0 wt % to 4 wt % |
| (PDMS) | |
| Flocculant (such as polyethylene oxide) | from 0 wt % to 1 wt % |
| Suds suppressor (such as silicone and/or fatty acid) | from 0 wt % to 5 wt % |
| Perfume (such as perfume microcapsule, spray-on perfume, starch | from 0 wt % to 1 wt % |
| encapsulated perfume accords, perfume loaded zeolite, and any | |
| combination thereof) | |
| Aesthetics (such as colorants) | from 0 wt % to 1 wt % |
| Miscellaneous | Balance |

Surfactant ingredients can be obtained from BASF, Ludwigshafen, Germany (Lutensol®); Shell Chemicals, London, UK; Stepan, Northfield, Ill., USA; Huntsman, Huntsman, Salt Lake City, Utah, USA; Clariant, Sulzbach, Germany (Praepagen®).

Sodium tripolyphosphate can be obtained from Rhodia, Paris, France. Zeolite can be obtained from Industrial Zeolite (UK) Ltd, Grays, Essex, UK. Citric acid and sodium citrate can be obtained from Jungbunzlauer, Basel, Switzerland. NOBS is sodium nonanoyloxybenzenesulfonate, supplied by Eastman, Batesville, Ark., USA.

TAED is tetraacetylethylenediamine, supplied under the Peractive® brand name by Clariant GmbH, Sulzbach, Germany.

Sodium carbonate and sodium bicarbonate can be obtained from Solvay, Brussels, Belgium.

Polyacrylate, polyacrylate/maleate copolymers can be obtained from BASF, Ludwigshafen, Germany.

Repel-O-Tex® can be obtained from Rhodia, Paris, France.

Texcare® can be obtained from Clariant, Sulzbach, Germany. Sodium percarbonate and sodium carbonate can be obtained from Solvay, Houston, Tex., USA.

Na salt of Ethylenediamine-N,N′-disuccinic acid, (S,S) isomer (EDDS) was supplied by Octel, Ellesmere Port, UK.

Hydroxy ethane di phosphonate (HEDP) was supplied by Dow Chemical, Midland, Mich., USA.

Enzymes Savinase®, Savinase® Ultra, Stainzyme® Plus, Lipex®, Lipolex®, Lipoclean®, Celluclean®, Carezyme®, Natalase®, Stainzyme®, Stainzyme® Plus, Termamyl®, Termamyl® ultra, and Mannaway® can be obtained from Novozymes, Bagsvaerd, Denmark.

Enzymes Purafect®, FN3, FN4 and Optisize can be obtained from Genencor International Inc., Palo Alto, Calif., US.

Direct violet 9 and 99 can be obtained from BASF DE, Ludwigshafen, Germany. Solvent violet 13 can be obtained from Ningbo Lixing Chemical Co., Ltd. Ningbo, Zhejiang, China. Brighteners can be obtained from Ciba Specialty Chemicals, Basel, Switzerland.

All percentages and ratios are calculated by weight unless otherwise indicated. All percentages and ratios are calculated based on the total composition unless otherwise indicated. It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Wash Assays

### Launder-O-Meter (LOM) Model Wash System

The Launder-O-Meter (LOM) is a medium scale model wash system that can be applied to test up to 20 different wash conditions simultaneously. A LOM is basically a large temperature-controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, and the more time consuming full scale experiments in front loader washing machines.

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM is basically a large temperature-controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time-consuming full-scale experiments in front loader washing machines.

In miniLOM, washes are performed in 50 ml test tubes placed in Stuart rotator.

### Terg-O-Tometer (TOM) Wash Assay

The Tergo-To-Meter (TOM) is a medium scale model wash system that can be applied to test 16 different wash conditions simultaneously. A TOM is basically a large temperature controlled water bath with up to 16 open metal beakers submerged into it. Each beaker constitutes one small top loader style washing machine and during an experiment, each of them will contain a solution of a specific detergent/enzyme/polymer system and the soiled and unsoiled fabrics its performance is tested on. Mechanical stress is achieved by a rotating stirring arm, which stirs the liquid within each beaker.

The TOM model wash system is mainly used in medium scale testing of detergents, enzymes and polymers at EU or AP wash conditions. In a TOM experiment, factors such as the ballast to soil ratio and the fabric to wash liquor ratio can be varied. Therefore, the TOM provides the link between small scale experiments, and the more time consuming full scale experiments.

Equipment: The water bath with 16 steel beakers and 1 rotating arm per beaker with capacity of 1 L detergent solution. Temperature ranges from 5° C. to 80° C. The water bath has to be filled up with deionised water. Rotational speed can be set up to 70 to 120 rpm/min.

Set temperature in the Terg-O-Tometer and start the rotation in the water bath. Wait for the temperature to adjust (tolerance is +1-0.5° C.). All beakers shall be clean and without traces of prior test material.

The wash solution with desired amount of detergent, temperature and water hardness is prepared in a bucket. The detergent is allowed to dissolve during magnet stirring for 10 min. Wash solution shall be used within 30 to 60 min after preparation.

1 L wash solution is added into a TOM beaker. The wash solution is agitated at 120 rpm and optionally one or more enzymes or polymers are added to the beaker. The swatches are sprinkled into the beaker and then the ballast load. Time measurement starts when the swatches and ballast are added to the beaker. The swatches are washed for 20 or 30 minutes after which agitation is terminated.

The wash load is subsequently transferred from the TOM beaker to a sieve and rinse with cold tap water. The soil swatches are separated from the ballast load. The soil swatches are transferred to a 5 L beaker with cold tap water under running water for 5 minutes. The ballast load is kept separately for the coming inactivation. The water is gently pressed out of the swatches by hand and placed on a tray covered with a paper. The swatches are allowed to dry overnight before subjecting the swatches to analysis, such as measuring the color intensity using a Digi-Eye as described herein.

### Full Scale Wash (FSW) Assay

FSW is used to evaluate wash performance in washing machines under scientifically designed conditions.

Wash conditions: Standard EU washing conditions are described below

| EU washing machine | |
|---|---|
| Miele WPS W5841 | Description |
| Wash program | Cotton/short |
| Water level | 15.6 L with Water plus |
| (water plus) | Standard EU conditions include activation |
| | of “Water Plus” (giving approx. |
| | 1 L water extra in the main wash). |
| Ballast | 4 kg total weight (including swatches) |
| | is standard. Mixed cotton/polyester ratio |
| | at 65/35 |
| Temperature | 30° C. |
| Wash time | 51 min |
| Water hardness | 15°dH. Ca²⁺/Mg²⁺/HCO₃⁻ Ca²⁺/Mg²⁺/HCO₃⁻ |
| | ratio 4:1:7.5 |
| Detergent | Detergent and dosage: according to the study |
| Enzyme | per study design, in wt. % of detergent |
| Test swatches | Real items like T-shirt, socks, pillowcase etc. |
| Soil | Pigment soil WFK09V |

Wash Procedure
a. Prepare the ballast and test swatches.
b. Select parameters for the wash: Program and Water level. Please note when using the automatic water injection, the declared water level is NOT the true water level. For 32 L program, the actual water intake is 33 L. The amount of enzyme and detergent added should be adjusted to reflect such water level.
c. Program and start automatic water meter system.
d. Press Start/Pause button to start water filling and adjust water temperature during the water intake. Water consumption is registered automatically during this time.
e. Add Ca/Mg according to water consumption.

a. Press the button to stir water.

b. Stop and check the water hardness.
f. The detergent and powder NaHCO₃ are added to the machine—short mix (10 sec).
g. The enzyme solution or granulate is added—short mix (10 sec).
h. Ballast and swatches are added to the washing machine.
i. Quickly Re-start the washing machine, select the wash program and water level, start the wash by pressing the Start/Pause button. If soaking, stir 30 s, then stay for the soaking time. The machine will automatically inject water (0.2-1 L) during main wash if low water level is detected.
j. Measure pH after 8 or 10 min in wash.
k. The washing machine by default gives 2 rinses. Rinsing with water having the same hardness. The machine will automatically add 1 more rinse if over foaming is detected. Water consumption is registered automatically during this time.
l. After the wash is completed, the test swatches are removed from the tea towels and placed on trays for drying—make sure the swatches are dried in completely darkness, as many stains are sensitive to light. The swatches are dried overnight and must be completely dry before measurement.

### Digi-Eye Measure Assay (Color Measurement)

The Digi-Eye is a controlled digital imaging system for measuring color difference and capturing repeatable images.

It is capable of obtaining reflectance or colorimetric data from samples.

The procedures described in this document, are our standard procedures.

The Digi-Eye has other functions, e.g.:
Measuring many samples with one click (using custom templates)
Measuring glossy samples (using “angled” illumination)
Measuring e.g. a stain without including the label in the measurement
Calculating the area of a certain color (e.g. % stained area)

Before measuring samples, check that a white calibration has been made within the past week, and that a colour calibration has been made within the last four hours. Set the Digi-eye as D65 and without UV. Put samples on the white measurement-plate and click the release-icon and then use the menu “DigiPix” to open the “Colour Measurement” window and measure all the selected area and then get the result Color difference R460.

### Enzyme Assays

### Assay I: Testing of DNase Activity

DNase activity is determined on DNase Test Agar with Methyl Green (BD, Franklin Lakes, N.J., USA), prepared according to the manual from supplier. Briefly, 21 g of agar is dissolved in 500 ml water and then autoclaved for 15 min at 121° C. Autoclaved agar is temperated to 48° C. in water bath, and 20 ml of agar is poured into Petri dishes with and allowed to solidify by incubation o/n at room temperature. On solidified agar plates, 5 μl of enzyme solutions are added, and DNase activity are observed as colorless zones around the spotted enzyme solutions.

### Methods

General methods of PCR, cloning, ligation nucleotides etc. are well-known to a person skilled in the art and may for example be found in in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

### EXAMPLES

### Example 1

Preparation of the Swatches:

New towels were purchase from IKEA with the product name “HAREN”, which made by 100% cotton with white color and the size is 40*70 cm. The towels were used by volunteers in daily life after face washing or shower for 2 weeks and the towels should be hung in the bathroom without direct sunlight environment. The towels were collected from the volunteers and the homogenous ones were selected for test.

The centre of towel was cut into 6 pieces of the size 10×10 cm and then the edges were sewn.

Washing Experiment:

Washing experiment were conducted in TOM wash. Specifically for EU condition water with hardness 15 dH (Ca:Mg:HCO₃=4:1:7.5) while AP condition water with hardness 14 dH (Ca:Mg=3:2), temperature 30 C, main wash time 20 min, 3.33 g/L model A detergent for EU or 2 g/L model X detergent for AP were conducted in TOM wash. The typical wash conditions are listed below in which 0.7 g/L soil (pigmentschmutz, 09V, wfk, Krefeld, Germany) was used in wash for anti-redeposition effect and different enzyme and polymer were added for comparison. For each of the six conditions below two pieces of towel from four different towels were used, i.e. each wash condition contained eight pieces of towels.

| | | | Color difference |
|---|---|---|---|
| Region | Detergent | Enzyme | vs Reference |
| EU | Reference: Model A | | |
| | detergent without | | |
| | polymer | | |
| | Model A detergent | | 3 |
| | with polymer | | |
| | Model A detergent | 0.8% of formulated | 3 |
| | without polymer | A. oryzae DNase^{a)} | |
| AP | Reference: Model X | | |
| | detergent without | | |
| | CMC | | |
| | Model X detergent | | 4 |
| | with CMC | | |
| | Model X detergent | 0.7% of formulated | 5 |
| | without CMC | A. oryzae DNase^{b)} | |

^{a)}Corresponding to 1 ppm active enzyme protein of SEQ ID NO: 14
^{b)}Corresponding to 0.5 ppm active enzyme protein of SEQ ID NO: 14

The results above show that the polymer can be replaced by DNase enzyme while maintaining the same level of whiteness.

1. A method of maintaining or improving whiteness of an item and/or preventing, reducing or removing redeposition of soil to an item during a wash cycle, the method comprising washing in the presence of a polypeptide having DNase activity and in the absence of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof, wherein the item is a textile.

2. A method of maintaining or improving whiteness of an item and/or preventing, reducing or removing redeposition of soil to an item during a wash cycle, the method comprising washing in the presence of a polypeptide having DNase activity as a reduction or even replacement for a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.

3. (canceled)

4. The method of claim 2, wherein the polypeptide having DNase activity is obtained from a fungal source, preferably Aspergillus, e.g., A. oryzae.

5. The method of claim 2, wherein the polypeptide having DNase activity is obtained from a bacterial source, preferably Bacillus, e.g. B. cibi.

6. The method of claim 2, wherein the polypeptide having DNase activity has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or a polypeptide having at least 60%, at least 65%, at least 70%, at least 75%, at least 80%, at least 85%, at least 90%, at least 95%, or even 100% sequence identity thereto.

7. The method of claim 2, which provides improved whiteness and/or improved anti-redeposition compared to use in the presence of the polyacrylic acid, modified polyacrylic acid polymer, modified polyacrylic acid copolymer, maleic acid-acrylic acid copolymer, carboxymethyl cellulose, cellulose gum, methyl cellulose, and/or combinations thereof.

8. The method of claim 2, wherein the detergent is a liquid detergent in the absence of a polyacrylic acid, a modified polyacrylic acid polymer, a modified polyacrylic acid copolymer, a maleic acid-acrylic acid copolymer, and/or combinations thereof.

9. The method of claim 2, wherein the detergent is a powder detergent in the absence of carboxymethyl cellulose, cellulose gum, methyl cellulose, and combinations thereof.

10. The method of claim 2, further comprising use of a polypeptide having cellulase activity.

11. The method of claim 10, wherein the polypeptide having cellulase activity has an amino acid sequence selected from the group consisting of SEQ ID NO: 10, SEQ ID NO: 11, SEQ ID NO: 12, SEQ ID NO: 13 or a polypeptide having at least 60%, at least 65%, at least 70%, at least 75%, at least 80%, at least 85%, at least 90%, at least 95%, or even 100% sequence identity thereto.

12. A detergent composition comprising a polypeptide having deoxyribonuclease (DNase) activity and a detergent adjunct ingredient, provided that the composition comprises less than 1%, e.g., less than 0.8%, less than 0.7%, less than 0.6%, less than 0.5%, less than 0.4%, less than 0.3%, less than 0.2%, less than 0.1%, less than 0.05%, less than 0,025% by weight of a polyacrylic acid, a modified polyacrylic acid polymer, a modified polyacrylic acid copolymer, a maleic acid-acrylic acid copolymer, carboxymethyl cellulose, cellulose gum, methyl cellulose, and/or combinations thereof.

13. The detergent composition of claim 12, wherein the polypeptide having deoxyribonuclease (DNase) activity is obtained from a fungal source, preferably Aspergillus, e.g., A. oryzae.

14. The detergent composition of claim 12, wherein the polypeptide is obtained from a bacterial source, preferably Bacillus, e.g., B. cibi.

15. The detergent composition of claim 12, wherein the polypeptide has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14 or a polypeptide having at least 60%, at least 65%, at least 70%, at least 75%, at least 80%, at least 85%, at least 90%, at least 95%, or even 100% sequence identity thereto.

16. The detergent composition of claim 12, which is a liquid detergent.

17. The detergent composition of claim 12, which is a powder detergent.

18. The detergent composition of claim 12, further comprising a polypeptide having cellulase activity.

19. The detergent composition of claim 18, wherein the polypeptide having cellulase activity has an amino acid sequence selected from the group consisting of SEQ ID NO: 10, SEQ ID NO: 11, SEQ ID NO: 12, SEQ ID NO: 13 or a polypeptide having at least 60%, at least 65%, at least 70%, at least 75%, at least 80%, at least 85%, at least 90%, at least 95%, or even 100% sequence identity thereto.


SEQUENCE LISTING (14 sequences)

SEQ ID NO: 1 - protein, 225 aa, Aspergillus oryzae
VPVNPEPDATSVENVALKTGSGDSQSDPIKADLEVKGQSALPFDVMCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALMFSLGSNDKSVCDKNKNIAGDWGFDPAKWAYQYDEKNNKFNYVGK

SEQ ID NO: 2 - protein, 109 aa, Bacillus licheniformis
ARYDDILYFPASRYPETGAHISDAIKAGHSDVCTIERSGADKRRQESLKGIPTKPGFDRDEWPMAMCEEGGKGASVRYVSSSDNRGAGSWVGNRLSGFADGTRILFIVQ

SEQ ID NO: 3 - protein, 110 aa, Bacillus subtilis
ASSYDKVLYFPLSRYPETGSHIRDAIAEGHPDICTIDRDGADKRREESLKGIPTKPGYDRDEWPMAVCEEGGAGADVRYVTPSDNRGAGSWVGNQMSSYPDGTRVLFIVQ

SEQ ID NO: 4 - protein, 245 aa, Serratia marcescens
DTLESIDNCAVGCPTGGSSNVSIVRHAYTLNNNSTTKFANWVAYHITKDTPASGKTRNWKTDPALNPADTLAPADYTGANAALKVDRGHQAPLASLAGVSDWESLNYLSNITPQKSDLNQGAWARLEDQERKLIDRADISSVYTVTGPLYERDMGKLPGTQKAHTIPSAYWKVIFINNSPAVNHYAAFLFDQNTPKGADFCQFRVTVDEIEKRTGLIIWAGLPDDVQASLKSKPGVLPELMGCKN

SEQ ID NO: 5 - protein, 182 aa, Bacillis Idriensis
LPPGTPSKSTAQSQLNALTVQTEGSMTGYSRDKFPHWISQGNGCDTRQVVLQRDADYYSGTCPVTSGKWYSYYDGVTLYNPSDLDIDHVVALAEAWRSGASSWTTDKREDFANDLSGTQLIAVSASTNRSKGDQDPSTWQPPRSGAACGYAKWWISTKYKWNLNLQSSEKTALQSMLNSCSY

SEQ ID NO: 6 - protein, 182 aa, Bacillus cibi [sequence omitted]

SEQ ID NO: 7 - protein, 182 aa, Bacillus horikoshii
LPPGTPSKSEAQSQLNSLTVKSEDPMTGYSRDHFPHWSGQGNGCDTRQIVLQRDADYYSGNCPVTSGKWYSYFDGVIVYSPSEIDIDHVVPLAEAWRSGASSWTTEQRRSFANDLNGPQLIAVTASVNRSKGDQDPSTWQPPRAGARCAYAKWWINTKHRWNLHLQSSEKSALQTMLNGCVY

SEQ ID NO: 8 - protein, 182 aa, Bacillus sp.
FPPEIPSKSTAQSQLNSLTVKSEDAMTGYSRDKFPHWISQGDGCDTRQMVLKRDADYYSGSCPVTSGKWYSYYDGITVYSPSEIDIDHIVPLAEAWRSGASSWTTEKRRNFANDLNGPQLIAVTASVNRSKGDQDPSTWQPPRSGARCAYAKMWVNTKYRWGLHLQSAEKSGLESMLNTCSY

SEQ ID NO: 9 - protein, 182 aa, Bacillus sp.
LPPGTPSKSEAQSQLTSLTVKPEDPMTGYSRDHFPHWISQGNGCNTRQIVLQRDADYYSGNCPVTTGKWYSYFDGVIVYSPSEIDIDHIVPLAEAWRSGASSWTAEQRRNFANDLNGPQLIAVTASVNRSKGDQDPSTWQPPRTGARCAYAKWWINTKYRWGLHLQSSEKSSLQSMLNGCAY

SEQ ID NO: 10 - protein, 415 aa, Humicola insolens
QKPGETKEVHPQLTTFRCTKRGGCKPATNFIVLDSLSHPIHRAEGLGPGGCGDWGNPPPKDVCPDVESCAKNCIMEGIPDYSQYGVTTNGTSLRLQHILPDGRVPSPRVYLLDKTKRRYEMLHLTGFEFTFDVDATKLPCGMNSALYLSEMHPTGAKSKYNPGGAYYGTGYCDAQCFVTPFINGLGNIEGKGSCCNEMDIWEANSRASHVAPHTCNKKGLYLCEGEECAFEGVCDKNGCGWNNYRVNVTDYYGRGEEFKVNTLKPFTVVTQFLANRRGKLEKIHRFYVQDGKVIESFYTNKEGVPYTNMIDDEFCEATGSRKYMELGATQGMGEALTRGMVLAMSIWWDQGGNMEWLDHGEAGPCAKGEGAPSNIVQVEPFPEVTYTNLRWGEIGSTYQEVQKPKPKPGHGPRSD

SEQ ID NO: 11 - protein, 773 aa, Bacillus akibai
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

SEQ ID NO: 12 - protein, 524 aa, Paenibacillus polymyxa
VVHGQTAKTITIKVDTFKDRKPISPYIYGTNQDLAGDENMAARRLGGNRMTGYNWENNMSNAGSDWQHSSDNYLCSNGGLTQAECEKPGAVVTSFHDQSLKLGTYSLVTLPMAGYVAADGNGSVQESEAAPSARWNQVVNAKNAPFQLQPDLNDNYVYVDEFVHFLVNKYGTASTKAGVKGYALDNEPALWSHTHPRIHPEKVGAKELVDRSVSLSKAVKAIDAGAEVFGPVLYGFGAYKDLQTAPDWDSVKGNYSWFVDYYLDQMRLSSQVEGKRLLDVFDVHWYPEAMGGGIRITNEVGNDETKKARMQAPRTLWDPTYKEDSWIAQWFSEFLPILPRLKQSVDKYYPGTKLAMTEYSYGGENDISGGIAMTDVLGILGKNDVYMANYWKLKDGVNNYVSAAYKLYRNYDGKNSTFGDTSVSAQTSDIVNSSVHASVTNASDKELHLVVMNKSMDSAFDAQFDLSGAKTYISGKVWGFDKNSSQIKEAAPITQISGNRFTYTVPPLTAYHIVLTTGNDTSPV

SEQ ID NO: 13 - protein, 214 aa, Melanocarpus albomyces
ANGQSTRYWDCCKPSCGWRGKGPVNQPVYSCDANFQRIHDFDAVSGCEGGPAFSCADHSPWAINDNLSYGFAATALSGQTEESWCCACYALTFTSGPVAGKTMVVQSTSTGGDLGSNHFDLNIPGGGVGLFDGCTPQFGGLPGARYGGISSRQECDSFPEPLKPGCQWRFDWFQNADNPSFTFERVQCPEELVARTGCRRHDDGGFAVFKAPSA

SEQ ID NO: 14 - protein, 221 aa, Aspergillus oryzae
VPVNPEPDATSVENVALKTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK